(19) United States
(12) Patent Application Publication
(10) Pub. No.: US 2025/0090383 A1
(43) Pub. Date: Mar. 20, 2025

(54) ENGINEERED KNITTED GARMENT HAVING INTEGRATED COMPRESSION ZONES

(71) Applicant: Lululemon Athletica Canada Inc., Vancouver (CA)

(72) Inventors: Audrey Milligan Reilly, Vancouver (CA); Sophie Adrienne Doyle, Vancouver (CA); Alison Claire Maxfield, Halfmoon Bay, CA (US); Joshua David Hiney, San Fransisco, CA (US); Yu Wen Angela Huang, Vancouver (CA); Sisi Jiang, Vancouver (CA); Eliot Jean Cohen Pirenne, Vancouver (CA)

(21) Appl. No.: 18/970,181

(22) Filed: Dec. 5, 2024

Related U.S. Application Data
(63) Continuation of application No. 17/006,177, filed on Aug. 28, 2020.
(60) Provisional application No. 62/894,292, filed on Aug. 30, 2019.

(51) Int. Cl. A61F 13/08; A41D 1/08

(52) U.S. Cl. CPC A61F 13/08; A41D 1/08; A41D 2500/10

(57) ABSTRACT

Examples of an engineered knitted garment with a number of integrated compression zones are disclosed. The engineered knitted garment may be a pair of tights having a first compression zone located at the calves, a second compression zone located at the knees, and a third compression zone located at the waist. The compressive moduli of the compression zones may be different to provide higher compression in areas where greater support is required (e.g., at the calves) and lower compression in areas where greater mobility is required (e.g., at the knees).

This application is a continuation of U.S. patent application Ser. No. 17/006,177, filed Aug. 28, 2020, entitled “ENGINEERED KNITTED GARMENT HAVING INTEGRATED COMPRESSION ZONES,” which application claims the benefit of and priority to U.S. Provisional Application Ser. No. 62/894,292, filed Aug. 30, 2019, entitled “ENGINEERED KNITTED GARMENT HAVING INTEGRATED COMPRESSION ZONES,” the disclosures of which are incorporated herein by reference in their entireties.

### TECHNICAL FIELD

This application relates to the field of garments, and more specifically, to an engineered knitted garment having integrated compression zones for sporting activities, exercise, and the like.

### BACKGROUND

Athletes and consumers are demanding high performance activewear and sportswear, such as for example, compression sportswear. Studies have found that compression garments can provide benefits such as reducing muscle fatigue, providing muscle support, reducing muscle vibration, reducing the risk of strain, preventing injuries, etc. Currently, most compression garments are made by cutting and sewing fabrics with different structural properties to provide different levels of compression. However, such garments typically have a number of seams located throughout the garment that may cause chaffing. In addition, making such garments typically involves greater fabric waste and increased labor and manufacturing costs.

### SUMMARY OF THE APPLICATION

In one aspect, an engineered knitted garment such as a pair of tights is provided having at least a first compression zone located at the calves, a second compression zone located at the knees, and a third compression zone located at the waist. The compressive moduli of the compression zones may be different to provide higher compression in areas where greater support is required (e.g., at the calves) and lower compression in areas where greater mobility is required (e.g., at the knees).

In addition to the aspects and embodiments described above, further aspects and embodiments will become apparent by reference to the drawings and study of the following detailed description.

### BRIEF DESCRIPTION OF THE DRAWINGS

Throughout the drawings, reference numbers may be re-used to indicate correspondence between referenced elements. The drawings are provided to illustrate example embodiments described herein and are not intended to limit the scope of the application. Sizes and relative positions of elements in the drawings are not necessarily drawn to scale. For example, the shapes of various elements and angles may be not drawn to scale, and some of these elements may be arbitrarily enlarged and positioned to improve drawing legibility.

FIG. 1 is a front view illustrating an example engineered knitted garment having integrated compression zones.

FIG. 2 is a rear view thereof.

FIG. 3 is a front view thereof illustrating example compression zones.

FIG. 4 is a rear view thereof.

FIG. 5 is a left side view illustrating an example knitted panel for the engineered knitted garment of FIGS. 1-4.

FIG. 6 is a front view illustrating another example engineered knitted garment having integrated mesh texture compression zones.

FIG. 7 is a rear view thereof.

FIG. 8 includes left, right, front, and rear views illustrating example knitted panels for the engineered knitted garment of FIGS. 6-7.

FIG. 9 is a block diagram illustrating an example warp knitting machine.

FIG. 10 is a front view illustrating another example engineered knitted garment having integrated compression zones.

FIG. 11 is a rear view thereof.

FIG. 12 is a front view illustrating another example engineered knitted garment having integrated compression zones.

FIG. 13 is a rear view thereof.

FIG. 14 is a front view illustrating another example engineered knitted garment having integrated compression zones.

FIG. 15 is a front view illustrating another example engineered knitted garment having integrated compression zones.

FIG. 16 is a front view illustrating another example engineered knitted garment having integrated compression zones.

FIG. 17 is a rear view thereof.

FIGS. 18-22 are front, side, and rear views illustrating additional example engineered knitted garments having integrated compression zones.

### DETAILED DESCRIPTION

In the following description, details are set forth to provide an understanding of the application. In some instances, certain structures, techniques, and methods have not been described or shown in detail in order not to obscure the application.

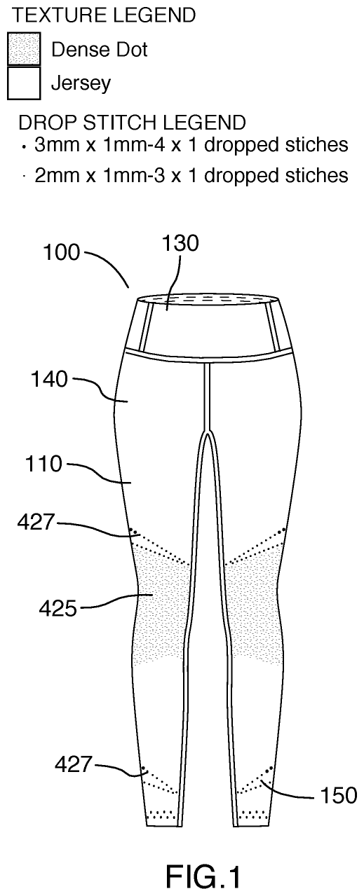

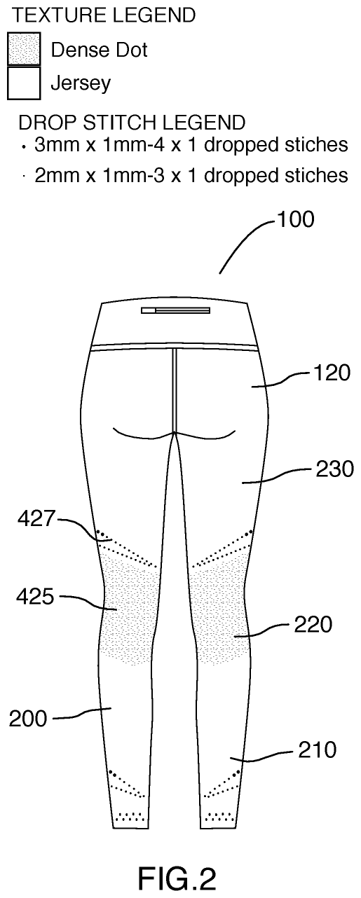

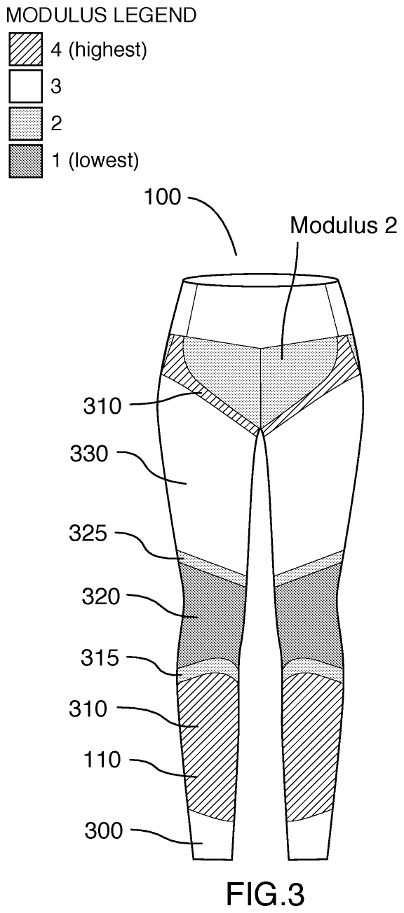

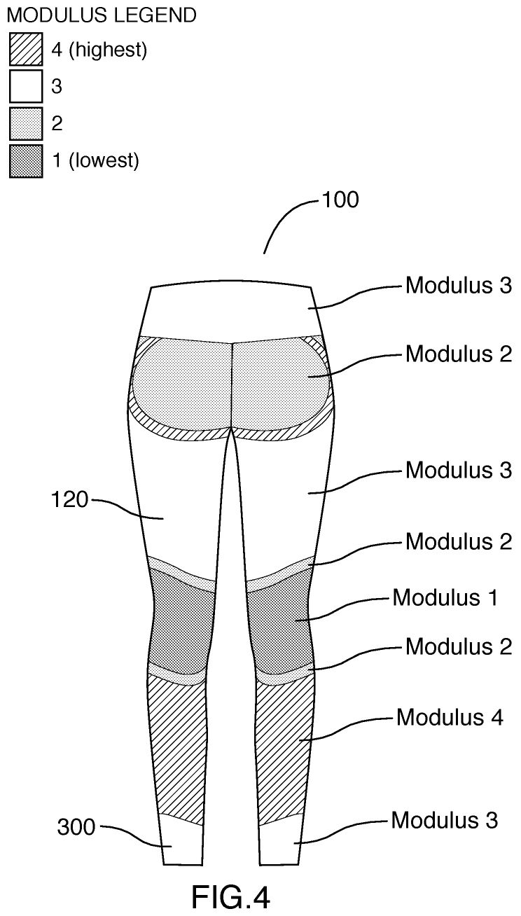

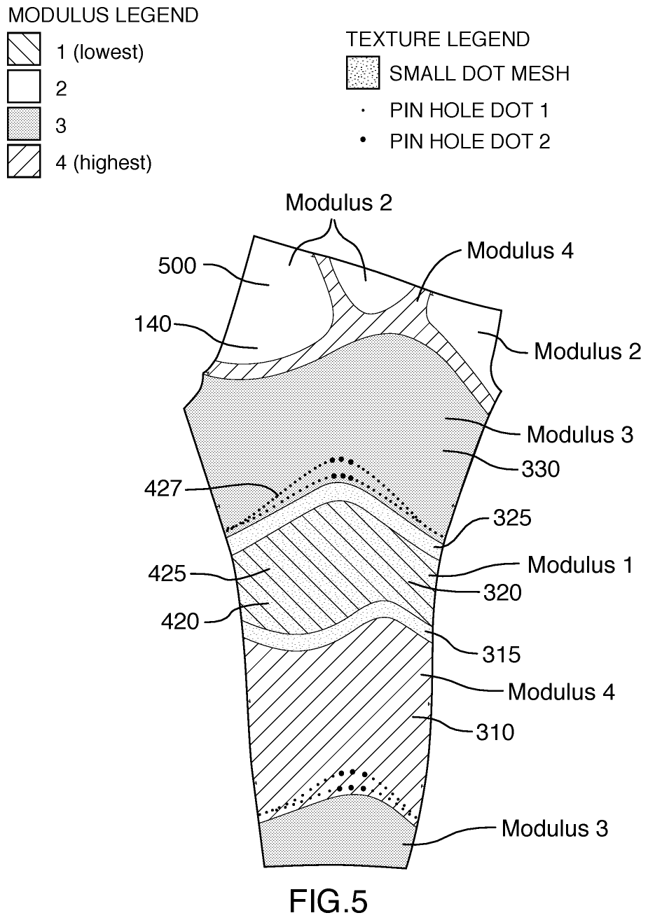

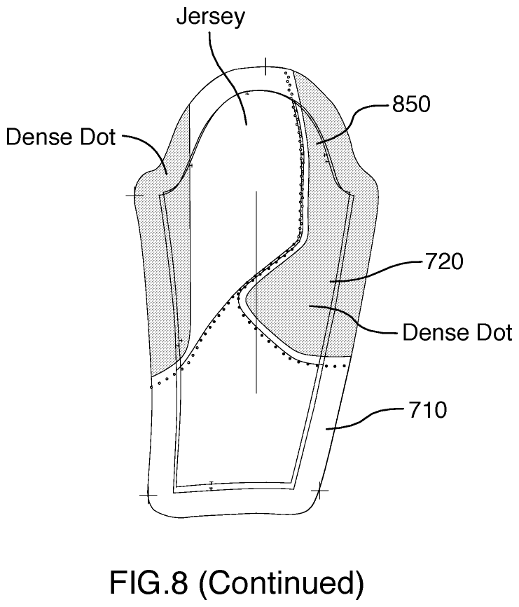

FIG. 1 is a front view illustrating an example engineered knitted garment 100 having integrated compression zones 300. FIG. 2 is a rear view thereof. FIG. 3 is a front view thereof illustrating example compressive zones 300. FIG. 4 is a rear view thereof. FIG. 5 is a left side view of an example knitted panel 500 for the engineered knitted garment 100 of FIGS. 1-4. And, FIG. 9 is a block diagram illustrating an example warp knitting machine 900.

According to one embodiment of the application, there is provided an engineered knitted garment 100 having integrated compression zones 300. The garment 100 may be a compression tight or pair of tights. Of course, the garment 100 may be any other garment such as a bra, top, shirt, jacket, skirt, skort, pants, shorts, bathing suits, etc. The garment 100 has a front side 110 and a rear side 120, a waistband 130, and first and second or left and right legs 140, 150. Each leg 140, 150 of the garment 100 may be formed from a knitted panel 500 (such as the left knitted panel 500 shown in FIG. 5).

The knitted panel 500 is shaped and sized to cover a portion of a wearer's body (e.g., the wearer's leg, a portion of the wearer's torso, etc.) and may be knitted using a raschel knitting machine 900 having two jacquard guide bars 910, 920. An example of such a machine is the Rascheltronic™ 4/2 jacquard raschel warp knitting machine available form Karl Mayer Holding GmbH & Co. KG. The two jacquard guide bars 910, 920 are used for seamlessly knitting different compression zones 300. One of the guide bars (e.g., 910) may be used to provide a desired combination of stiches and knit constructions 200 while the other guide bar (e.g., 920) may be used to adds extra yarn (e.g., jacquards in elastane) to change the density and/or weight in a particular zone such that the weight difference between the lowest compression zone and the highest compression zone may be around 200 grams per square meter (GSM). Typically, this cannot be obtained with a single jacquard bar knitting machine. Different levels (e.g., 3 or 4) of elastane content (or similar synthetic fibers or yarn such as Spandex™, Lycra™, polyamide, polyether-polyurea copolymer, etc.) may be engineered into the fabric of the knitted panel 500 to create different compression zones (e.g., 3 or 4) 300. Mesh textures 425 for ventilation zones 420 may also be included. The mesh textures 425 may be used to provide lower compression zones 300 in selected areas. Further compression zones 300 may be created by combining elastane jacquard with jacquard patterns which provides a double effect with respect to compression.

According to one embodiment, the knitted panel 500 may include a first compression zone 310 having a first knit stitch construction (or pattern) (e.g., Jersey knit, etc.) 210. One of the jacquard guide bars 910 of the knitting machine 900 may be used to define the first knit stitch construction 210 and the other of the jacquard guide bars 920 may be used to add an elastane jacquard construction (or pattern) to the first knit stitch construction 210 to increase fabric weight in the first compression zone 310. The first compression zone 310 may have a first amount of compression (or compressive modulus).

The knitted panel 500 may include a second compression zone 320 joined to the first compression zone 310 by a seamless transition 315 and may have a second knit stitch construction (or pattern) (e.g., Dense Dot knit, etc.) 220. The second compression zone 320 may have a second amount of compression (or compressive modulus). According to one embodiment, the first amount of compression of the first compression zone 310 may be greater than the second amount of compression of the second compression zone 320. That is, the first compression zone 310 may provide a tighter or more compressive fit that the second compression zone 320. Advantageously, the seamless joining of the first and second compression zones 310, 320 reduces chaffing.

According to one embodiment, a difference in fabric weight between the first compression zone 310 and the second compression zone 320 may be between 100 and 220 grams per square meter (GSM). According to one embodiment, the first and second knit stitch constructions 210, 220 of the garment 100 may be the same. According to one embodiment, at least one of the first and second knit stitch constructions 210, 220 may be a jacquard pattern.

For example, the second knit stitch construction 220 may include an elastane jacquard construction (or pattern) that is added to the second knit stitch construction 220 using the other of the jacquard guide bars. According to one embodiment, an amount (or extent) of the elastane jacquard in the first knit stitch construction 210 may be greater than an amount (or extent) of the elastane jacquard in the second knit stitch construction 220.

According to one embodiment, the engineered knitted garment 100 may further include a third compression zone 330 joined to at least one of the first compression zone 310 and the second compression zone 320 by a respective seamless transition 325 and having a third knit stitch construction (or pattern) (e.g. Jersey knit, etc.) 230. One of the jacquard guide bars of the knitting machine may be used to define the third knit stitch construction 230. The third compression zone 330 may have a third amount of compression (or compressive modulus). According to one embodiment, the third amount of compression of the third compression zone 330 may be greater than the second amount of compression of the second compression zone 320 but less than the first amount of compression of the first compression zone 310. The third knit stitch construction 230 may be a jacquard pattern. The third compression zone 330 may include an elastane jacquard construction (or pattern) added to the third knit stitch construction 230 using the other of the jacquard guide bars. According to one embodiment, an amount (or extent) of the elastane jacquard in the third compression zone 330 may be less than an amount of elastane jacquard in the first compression zone 310.

According to one embodiment, at least a portion of the second compression zone 320 may include a mesh texture or pattern 425. According to another embodiment, the garment 100 may further include at least one ventilation zone 420 seamlessly knitted into the knitted panel 500. The ventilation zone 420 may be formed from or include a mesh 425 or a pattern of drop stitch open holes 427. Advantageously, sheer and open hole mesh textures 425, 427 may be knit into the knitted panel 500 to improve breathability, to achieve a lower compressive modulus, to reduce restriction, and/or for visual effect.

As mentioned above, according to one embodiment as shown in FIGS. 1-4, the engineered knitted garment 100 may be a compression tight (or pair of compression tights or tights). In this embodiment, the first compression zone 310 may be located in one or more of a calf area, an under buttock area, and an upper waistband area with anchoring portions to respective hip areas of the compression tight 100. The second compression zone 320 may be located in a knee area of the compression tight 100. The garment 100 may further include a third compression zone 330 joined to at least one of the first compression zone 310 and the second compression zone 320 by a respective seamless transition 325 and having a third knit stitch construction 230 wherein one of the jacquard guide bars is used to define the third knit stitch construction 230, the third compression zone 330 having a third amount of compression (or compressive modulus), the third amount of compression being greater than the second amount of compression of the second compression zone 320 but less than the first amount of compression of the first compression zone 310. The third compression zone 330 may be located in one or more of a pelvic area and an area at least partially surrounding a knee area of the compression tight 100. Advantageously, the compressive moduli of the compression zones 300 may be different to provide higher compression in areas where greater support is required (e.g., at the calves) and lower compression in areas where greater mobility is required (e.g., at the knees). The high modulus in the under buttock and upper waistband areas anchors the compression tight 100 to the waistband and reduces crotch sagging and bum jiggle. The low modulus in the knee area of the compression tight 100 reduces restriction and bulk. And, the gradate levels of compression in the transition 325 provide a distraction free fit.

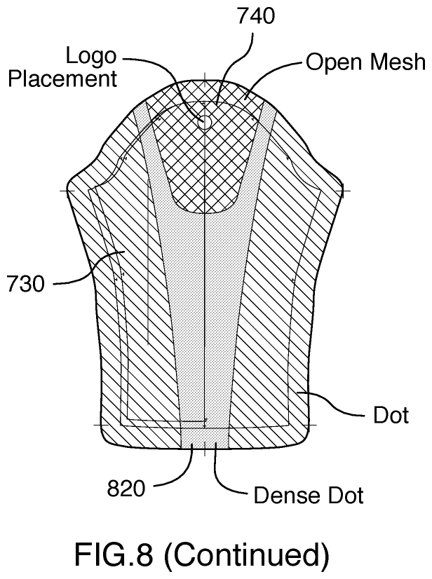

In some embodiments, the garment 100 may have more than three compression zones 300. For example, FIGS. 3 and 4 illustrate a garment 100 (e.g., a pair of tights) having 4 compression zones. As described further below, FIGS. 10 to 13 also illustrate garments 1000, 1200 having 4 compression zones.

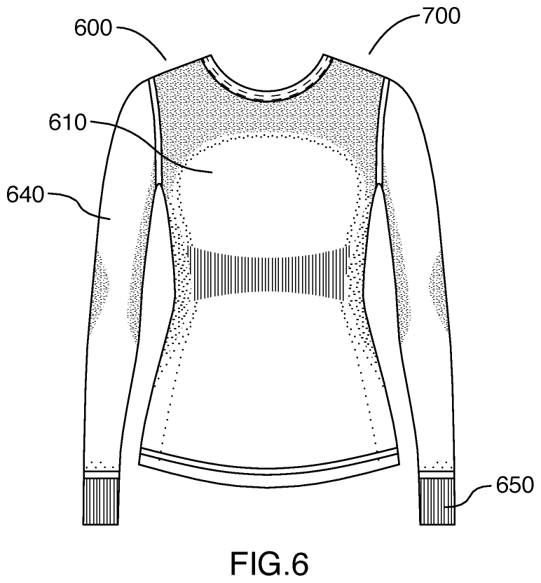

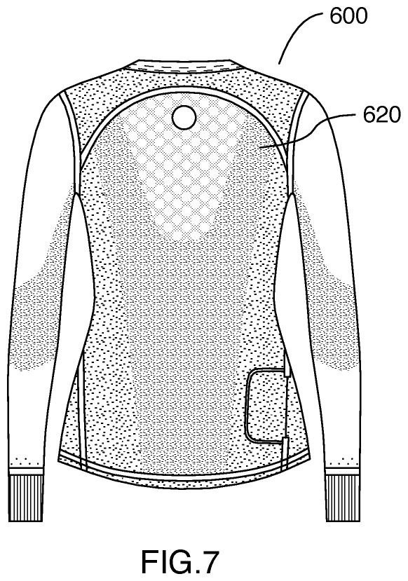

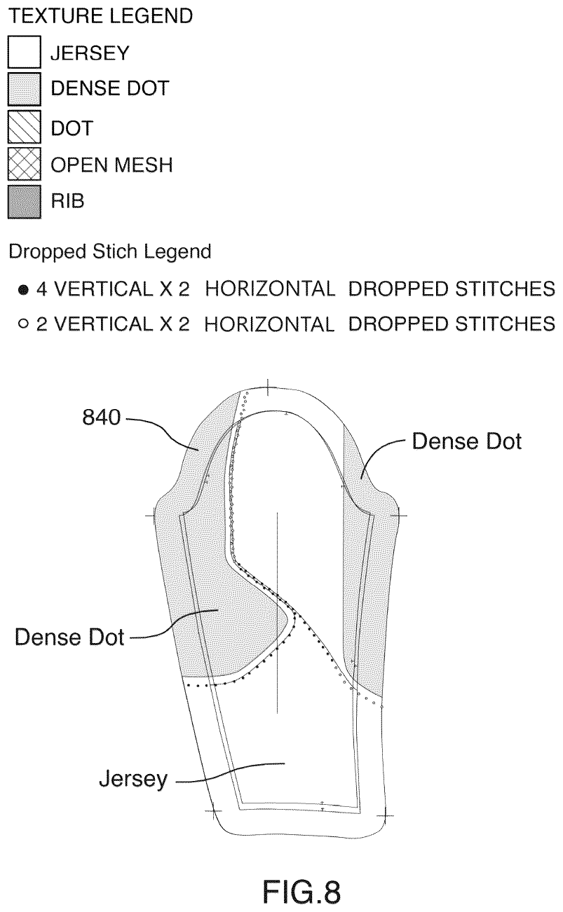

FIG. 6 is a front view illustrating another example engineered knitted garment 600 having integrated mesh texture compression zones 700. FIG. 7 is a rear view thereof. And, FIG. 8 includes left, right, front, and rear views illustrating example knitted panels 800 for the engineered knitted garment 600 of FIGS. 6-7.

As mentioned above, the engineered knitted garment of the present application may be any garment such as a bra, top, shirt, jacket, skirt, skort, pants, shorts, bathing suits, etc., in addition to the tights 100 shown in FIGS. 1-4. For example, FIG. 6 shows an engineered knitted garment 600 which is a top or shirt. The garment 600 has a front side 610, a rear side 620, and first and second or left and right arms 640, 650. Each arm 640, 650 and the front and rear sides 610, 620 of the garment 600 may be formed from a respective knitted panel 800 (such as the knitted panels 810, 820, 840, 850 shown in FIG. 8).

The garment 600 has multiple compression zones 700 that are formed from different mesh textures. The different mesh textures provide different levels of breathability (or ventilation) and different levels (or moduli) of compression. For example, the wrist area of each arm 640, 650 and the lower area on the front side 610 of the garment 600 may have a jersey texture providing a first compression zone 710. The elbow area of each arm 640, 650, the under neck area on the front side 610, and the middle back area on the rear side 620 of the garment 600 may have a dense dot texture providing a second compression zone 720. The underarm areas on the sides of the rear side 620 of the garment 600 may have a dot texture providing a third compression zone 730. The upper back area on the rear side 620 of the garment 600 may have an open mesh texture providing a fourth compression zone 740. And, the middle area on the front side 610 of the garment 600 may have a rib texture providing a fifth compression zone 750. For example, the first compression zone 710 (at the wrists) may have a higher compressive modulus than the second compression zone 720 (at the elbows) to improve fit and user comfort.

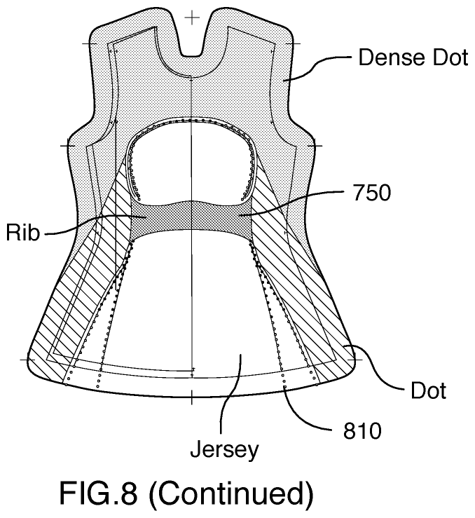

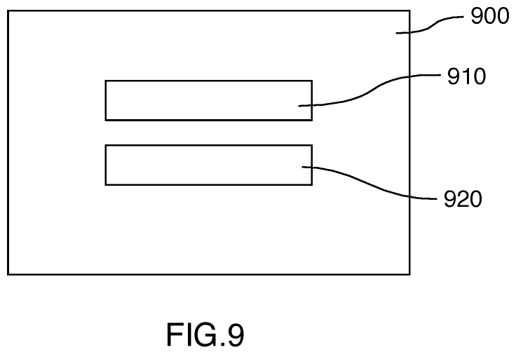

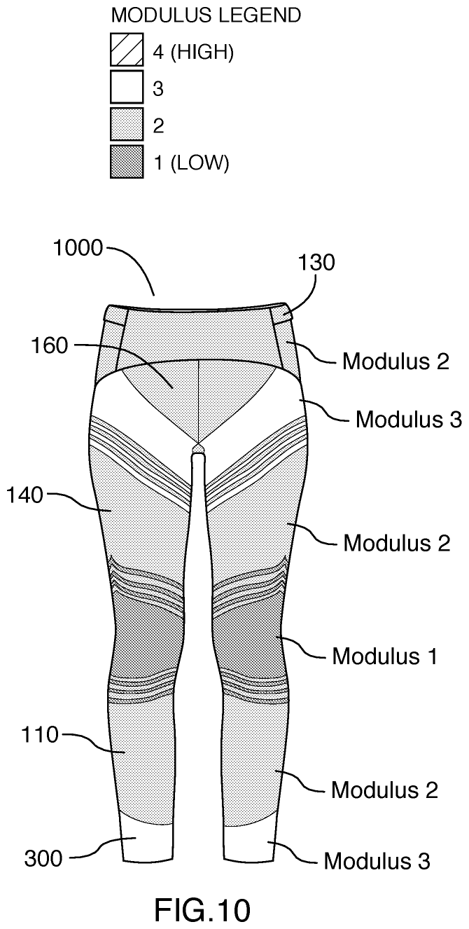

FIG. 10 is a front view illustrating another example engineered knitted garment 1000 having integrated compression zones 300. FIG. 11 is a rear view thereof. FIG. 12 is a front view illustrating another example engineered knitted garment 1200 having integrated compression zones 300. And, FIG. 13 is a rear view thereof. Similar to the engineered knitted garment 100 of FIGS. 3-4, the engineered knitted garments 1100, 1200 of FIGS. 10-11 and FIGS. 12-13 may be compression tights or pairs of tights for women and men, respectively. The garments 1100, 1200 each have a front side 110, a rear side 120, a waistband 130, and first and second or left and right legs 140, 150. The garments 1100, 1200 include a number (e.g., 4) of compression zones 300 each having a respective amount of compression (or compressive modulus) (e.g., Modulus 1, Modulus 2, etc.). The layout or arrangement of the compression zones 300 in the pelvic area 160 of the garments 1100, 1200 is tuned to match the sex of the wearer to provide support for sensitive areas. For example, and referring to FIG. 10 which shows the women's garment 1000, less support (i.e., a lower compressive modulus Modulus 2) may be provided in the genital area and greater support (i.e., a higher compressive modulus Modulus 3) may be provided around the genital area. As another example, and referring to FIG. 12 which shows the men's garment 1200, greater support (i.e., a higher compressive modulus Modulus 4) may be provided in the genital area and less support (i.e., a lower compressive modulus Modulus 2) may be provided around the genital area. The seamless transitions 1015, 1025 between compression zones 300 may be formed by alternating bands (e.g., 3 bands) of fabric from each of the adjoining compression zones 300. The garment 1100, 1200 may be of light weight being made from, for example, lighter 60 dtex elastane rather than, for example, the heavier 78 dtex elastane that may be used in the garment 100 of FIGS. 3-4. This construction is advantageous for use in running activities.

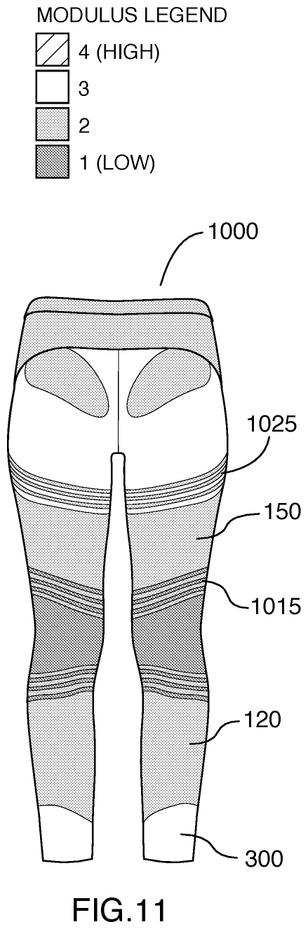

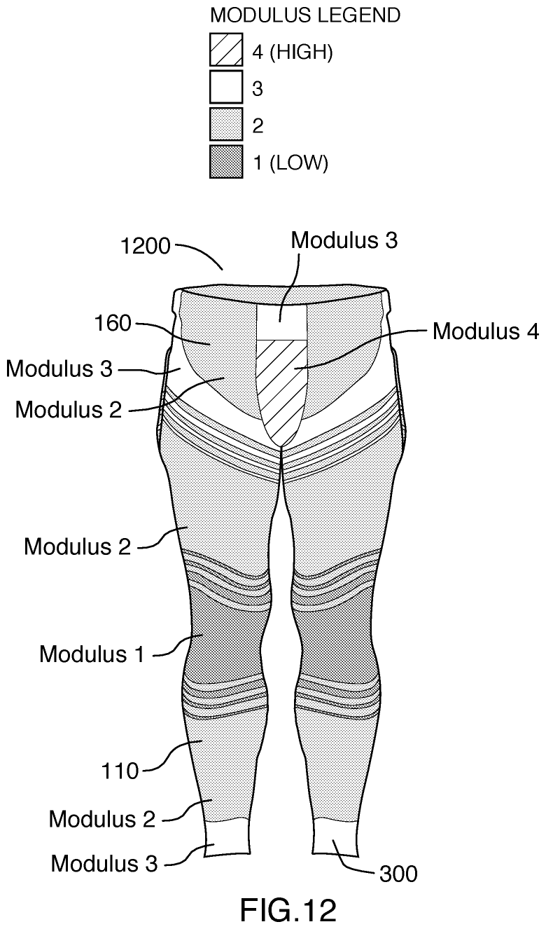

FIG. 14 is a front view illustrating another example engineered knitted garment 1400 having integrated compression zones 300. And, FIG. 15 is a front view illustrating another example engineered knitted garment 1500 having integrated compression zones 300. The engineered knitted garment 1400 of FIG. 14 may be a sports bra or top and the engineered knitted garment 1500 of FIG. 15 may be compression tights or a pair of tights. The garments 1400, 1500 include a number (e.g., 3) of compression zones 300 each having a respective amount of compression (or compressive modulus) (e.g., Modulus 1, Modulus 2, etc.). The layout or arrangement of the compression zones 300 in the breast area 170 of the garment 1400 of FIG. 14 is tuned to provide support for sensitive areas. For example, greater support (i.e., a higher compressive modulus Modulus 3) may be provided in the areola/nipple area, and on either side of the breast tissue, and less support (i.e., a lower compressive modulus Modulus 1) may be provided around the areola/nipple area.

The layout of the compression zones 300 in the joint areas 180 (e.g., in the knee area) of the garment 1500 of FIG. 15 is tuned to provide greater support (i.e., a higher compressive modulus Modulus 3) around the joint and less support (i.e., a lower compressive modulus Modulus 1) at the joint. This provides the wearer with an increased feeling of support and awareness in the joint areas 180. Such garments may be used in training activities where additional support around the joints is desired. The garments 1400, 1500 may be of light weight and may be made using cooling yarn which is advantageous for use in training activities.

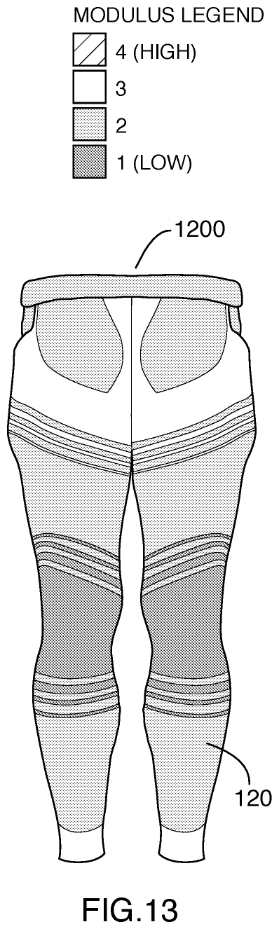

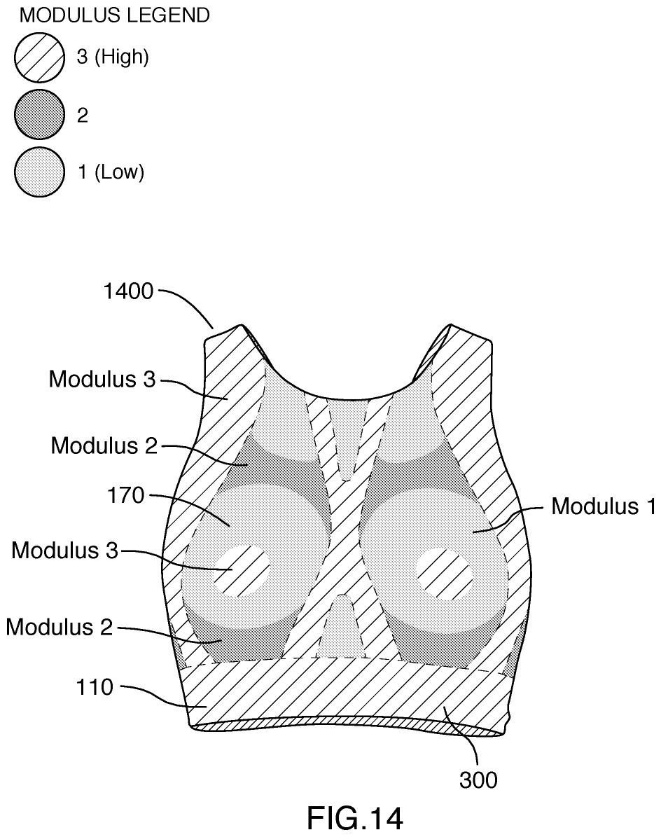

FIG. 16 is a front view illustrating another example engineered knitted garment 1600 having integrated compression zones 300. And, FIG. 17 is a rear view thereof. The engineered knitted garment 1600 of FIGS. 16-17 be a bathing suit or leotard. The garment 1600 includes a number (e.g., 3 or 4) of compression zones 300 each having a respective amount of compression (or compressive modulus) (e.g., Modulus 1, Modulus 2, etc.). The layout or arrangement of the compression zones 300 in the abdominal area 190 of the garment 1600 of FIG. 16 is tuned to provide support where required. For example, to improve flexibility, less support (i.e., a lower compressive modulus Modulus 2) may be provided in the central area of the abdomen and greater support (i.e., a higher compressive modulus Modulus 3) may be provided around the central area.

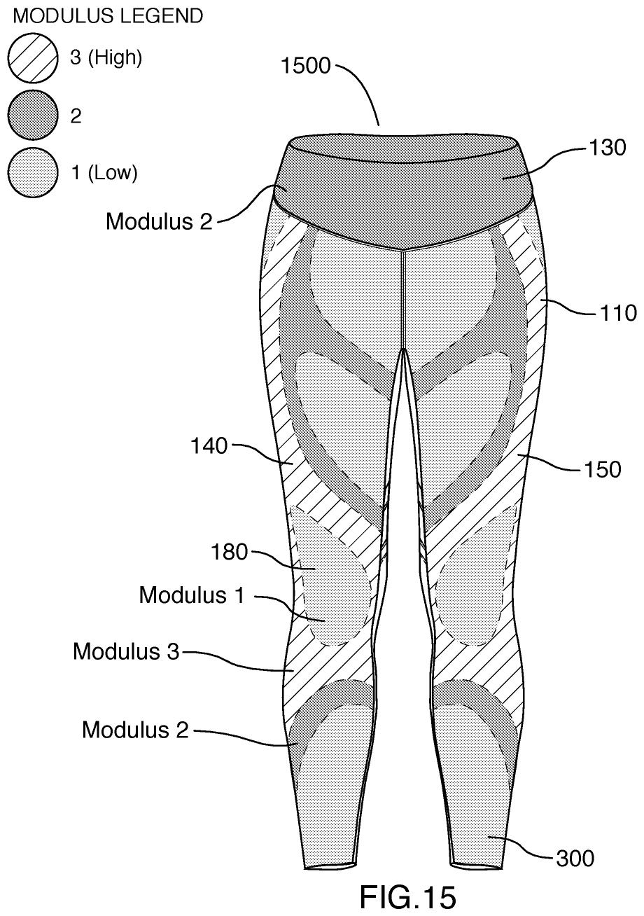

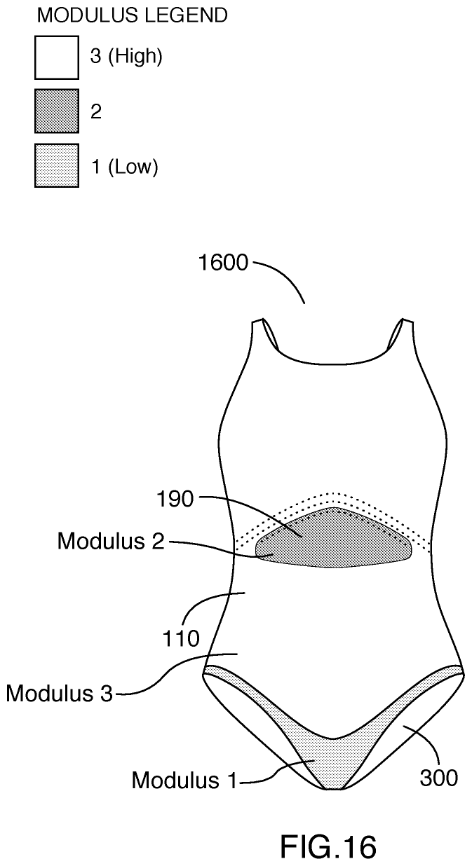

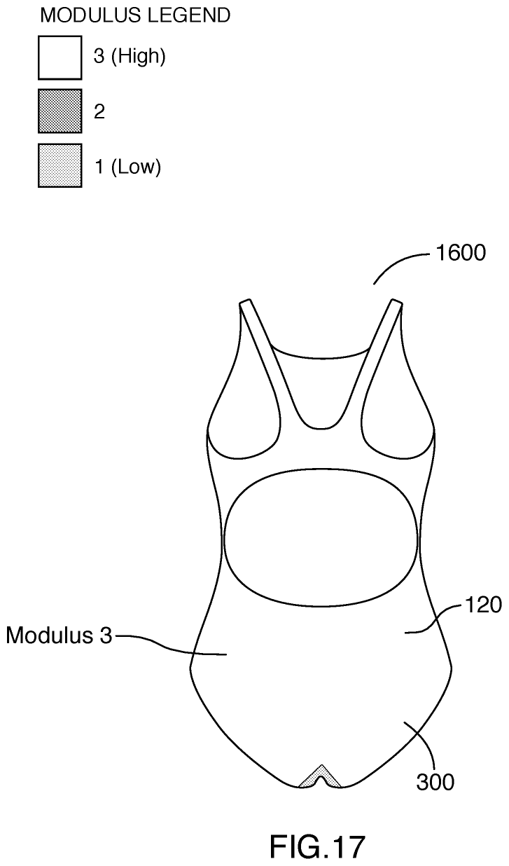

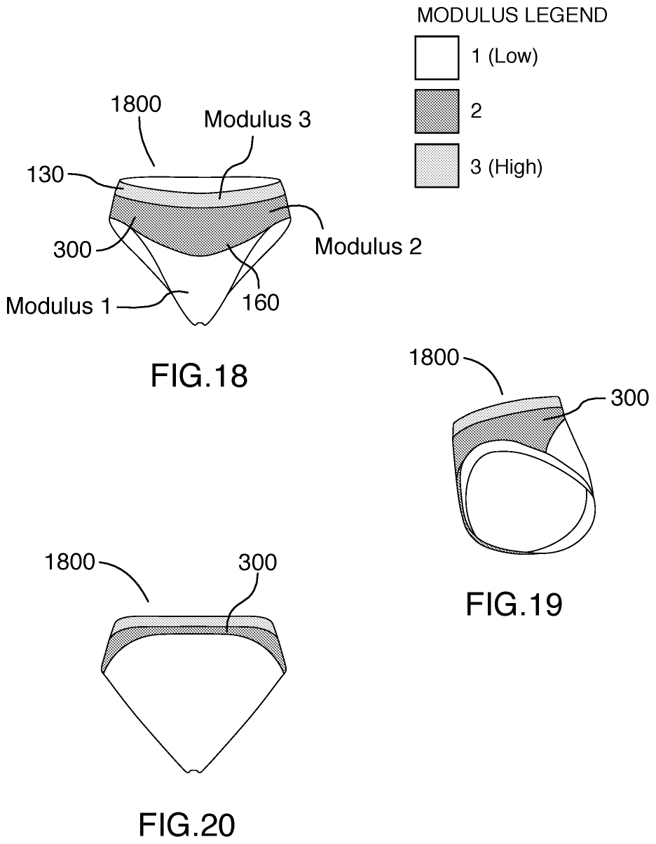

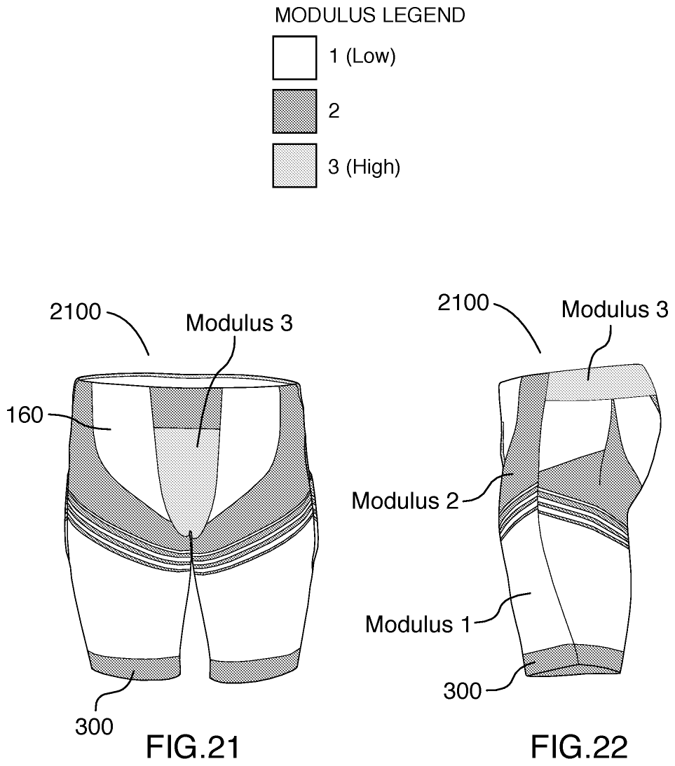

FIGS. 18-22 are front, side, and rear views illustrating additional example engineered knitted garments 1800, 2100 having integrated compression zones 300. The engineered knitted garments 1800, 2100 of FIGS. 18-20 and FIGS. 21-22 may be underwear for women and men, respectively. The garments 1800, 2100 include a number (e.g., 3 or 4) of compression zones 300 each having a respective amount of compression (or compressive modulus) (e.g., Modulus 1, Modulus 2, etc.). Similar to the garments 1100, 1200 of FIGS. 10-13, the layout or arrangement of the compression zones 300 in the pelvic area 160 of the garments 1800, 2100 is tuned to match the sex of the wearer to provide support where required for sensitive areas. In addition, and referring to FIGS. 18-20 which show the women's garment 1800, greater support (i.e., a higher compressive modulus Modulus 3) may be provided in the waistband area or waistband 130 to replicate or approximate the hugged feeling provided by a pair of compression tights and less support (i.e., a lower compressive modulus Modulus 2) may be provide in the pelvic area 160 below the waistband 130.

In some embodiments, the compression zones 300 may be made from a blend of natural and synthetic fabrics.

While particular elements, embodiments and applications of the present application have been shown and described, it will be understood, that the scope of the application is not limited thereto, since modifications can be made by those skilled in the art without departing from the scope of the present application, particularly in light of the foregoing teachings. Thus, for example, in any method or process disclosed herein, the acts or operations making up the method/process may be performed in any suitable sequence and are not necessarily limited to any particular disclosed sequence. Elements and components can be configured or arranged differently, combined, and/or eliminated in various embodiments. The various features and processes described above may be used independently of one another, or may be combined in various ways. All possible combinations and subcombinations are intended to fall within the scope of this application. Reference throughout this disclosure to “some embodiments,” “an embodiment,” or the like, means that a particular feature, structure, step, process, or characteristic described in connection with the embodiment is included in at least one embodiment. Thus, appearances of the phrases “in some embodiments,” “in an embodiment,” or the like, throughout this disclosure are not necessarily all referring to the same embodiment and may refer to one or more of the same or different embodiments. Indeed, the novel methods and systems described herein may be embodied in a variety of other forms; furthermore, various omissions, additions, substitutions, equivalents, rearrangements, and changes in the form of the embodiments described herein may be made without departing from the spirit of the application.

Various aspects and advantages of the embodiments have been described where appropriate. It is to be understood that not necessarily all such aspects or advantages may be achieved in accordance with any particular embodiment. Thus, for example, it should be recognized that the various embodiments may be carried out in a manner that achieves or optimizes one advantage or group of advantages as taught herein without necessarily achieving other aspects or advantages as may be taught or suggested herein.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without operator input or prompting, whether these features, elements and/or steps are included or are to be performed in any particular embodiment. No single feature or group of features is required for or indispensable to any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

The example calculations, simulations, results, graphs, values, and parameters of the embodiments described herein are intended to illustrate and not to limit the disclosed embodiments. Other embodiments can be configured and/or operated differently than the illustrative examples described herein.

What is claimed is:

1. A garment, comprising:
a pair of tights having a front side and a rear side, wherein the front side is sized and shaped to cover an area corresponding to a front portion of a wearer's torso and legs, wherein the rear side is sized and shaped to cover an area corresponding to a rear portion of a wearer's torso and legs, wherein the pair of tights has a left leg and a right leg, wherein the pair of tights is an engineered warp knitted garment having a plurality of integrated compression zones, the plurality of integrated compression zones including:
a first compression zone sized and shaped to cover a first area corresponding to at least a portion of a wearer's waist or torso, wherein the first compression zone extends from the front side to the rear side of the pair of tights, and wherein the first compression zone has a first amount of compression and a first fabric weight;
a second compression zone on the front side below the first compression zone, wherein the second compression zone is sized and shaped to cover at least a portion of a second area corresponding to a wearer's front torso below the waist, and wherein the second compression zone has a second amount of compression and a second fabric weight;
a third compression zone adjacent to the second compression zone, wherein the third compression zone is sized and shaped to cover at least a portion of a third area corresponding to at least a front side of a wearer's right and left hips below a waist, and wherein the third compression zone has a third amount of compression and a third fabric weight;
a fourth compression zone adjacent to the third compression zone and in each of the left leg and the right leg, wherein the fourth compression zone is sized and shaped to cover at least a portion of a fourth area corresponding to a wearer's torso at or below a hip and a wearer's leg above a knee, wherein the fourth compression zone extends around the front side and around the rear side of each of the left leg and the right leg, and wherein the fourth compression zone has a fourth amount of compression and a fourth fabric weight;
a fifth compression zone below the fourth compression zone in each of the left leg and the right leg, wherein the fifth compression zone is sized and shaped to cover at least a portion of a fifth area corresponding to a wearer's leg directly above a knee, wherein the fifth compression zone extends around the front side and around the rear side of each of the left leg and the right leg, and wherein the fifth compression zone has a fifth amount of compression and a fifth fabric weight;
a sixth compression zone below the fifth compression zone in each of the left leg and the right leg, wherein the sixth compression zone is sized and shaped to cover at least a portion of a sixth area corresponding to a wearer's knee, wherein the sixth compression zone extends around the front side and around the rear side of the left leg and the right leg, and wherein the sixth compression zone has a sixth amount of compression and a sixth fabric weight;
a seventh compression zone below the sixth compression zone in each of the left leg and the right leg, wherein the seventh compression zone is sized and shaped to cover at least a portion of a seventh area corresponding to a wear's leg directly below a knee, wherein the seventh compression zone extends around the front side and around the rear side of each of the left leg and the right leg, and wherein the seventh compression zone has a seventh amount of compression and a seventh fabric weight;
an eighth compression zone below the seventh compression zone in each of the left leg and the right leg, wherein the eighth compression zone is sized and shaped to at least partially cover an eighth area corresponding to a wearer's calf, wherein the eighth compression zone extends around the front side and around the rear side of each of the left leg and the right leg, and wherein the eighth compression zone has an eighth amount of compression and an eighth fabric weight; and
a ninth compression zone below the eighth compression zone in each of the left leg and the right leg, wherein the ninth compression zone is sized and shaped to cover at least a portion of a ninth area corresponding to at least a portion of a wearer's calf above an ankle or at least a portion of a wearer's ankle, wherein the ninth compression zone extends around the front side and around the rear side of each of the left leg and right leg, and wherein the ninth compression zone has a ninth amount of compression and a ninth fabric weight;
wherein the first compression zone is directly or indirectly joined to the second compression zone by a first seamless transition, the second compression zone is directly or indirectly joined to the third compression zone by a second seamless transition, the third compression zone is directly or indirectly joined to the fourth compression zone by a third seamless transition, the fourth compression zone is directly or indirectly joined to the fifth compression zone by a fourth seamless transition, the fifth compression zone is directly or indirectly joined to the sixth compression zone by a fifth seamless transition, the sixth compression zone is directly or indirectly joined to the seventh compression zone by a sixth seamless transition, the seventh compression zone is directly or indirectly joined to the eighth compression zone by a seventh seamless transition, and the eighth compression zone is directly or indirectly joined to the ninth compression zone by an eighth seamless transition;
wherein the first amount of compression is greater than the second amount of compression, the second amount of compression is less than the third amount of compression, the fourth amount of compression is greater than the fifth amount of compression, the fifth amount of compression is greater than the sixth amount of compression, the sixth amount of compression is less than the seventh amount of compression, the seventh amount of compression is less than the eighth amount of compression, or the eighth amount of compression is greater than the ninth amount of compression;
wherein a difference between the sixth fabric weight and the third fabric weight ranges from 100 grams per square meter (GSM) to 220 GSM, and wherein the third fabric weight is greater than the sixth fabric weight; and
wherein at least one compression zone of the first compression zone, the second compression zone, the third compression zone, the fourth compression zone, the fifth compression zone, the sixth compression zone, the seventh compression zone, the eighth compression zone, and the ninth compression zone includes
a jacquard knit stitch construction, and
an elastane jacquard construction added in the jacquard knit stitch construction, wherein the elastane jacquard construction is configured to increase a density or configured to increase a fabric weight or configured to increase both a density and a fabric weight.

2. The garment of claim 1, wherein the plurality of integrated compression zones further comprise
a tenth compression zone on the rear side of the pair of tights, wherein the tenth compression zone is sized and shaped to cover a tenth area corresponding to a rear side of a wearer's torso below the waist, wherein the tenth compression zone has a tenth amount of compression and a tenth fabric weight; and
an eleventh compression zone on the rear side adjacent to the tenth compression zone, wherein the eleventh compression zone is sized and shaped to cover an eleventh area corresponding to a rear size of a wearer's torso adjacent to the tenth area, and wherein the eleventh compression zone has an eleventh amount of compression and an eleventh fabric weight;
wherein the first compression zone is directly or indirectly joined to the tenth compression zone by a ninth seamless transition, and the tenth compression zone is directly or indirectly joined to the eleventh compression zone by a tenth seamless transition; and wherein a difference between the sixth fabric weight and the eleventh fabric weight ranges from 100 grams per square meter (GSM) to 220 GSM, wherein the eleventh fabric weight is greater than the sixth fabric weight.

3. The garment of claim 2, wherein at least two compression zones of the first compression zone, the second compression zone, the third compression zone, the fourth compression zone, the fifth compression zone, the sixth compression zone, the seventh compression zone, the eighth compression zone, the ninth compression zone, the tenth compression zone, and the eleventh compression zone each include the jacquard knit stitch construction.

4. The garment of claim 1, wherein the first compression zone, the third compression zone, and the fourth compression zone each include the jacquard knit stitch construction.

5. The garment of claim 1, wherein at least two compression zones of the first compression zone, the third compression zone, the fourth compression zone, the eighth, and the ninth compression zone each include the jacquard knit stitch construction.

6. The garment of claim 1, wherein the third compression zone is joined directly to the first compression zone and to the second compression zone, and the third compression zone includes anchoring portions at or below a waistband of the pair of tights.

7. The garment of claim 1, wherein the third amount of compression is about the same as the eighth amount of compression.

8. The garment of claim 1, wherein the first amount of compression is about the same as the fourth amount of compression, or the first amount of compression is about the same as the eighth amount of compression, or the first amount of compression, the fourth amount of compression, and the eighth amount of compression are each individually about the same.

9. The garment of claim 1, wherein the fifth amount of compression is about the same as the seventh amount of compression, or wherein the second amount of compression, the fifth amount of compression, and the seventh amount of compression are each individually about the same.

10. The garment of claim 1, wherein at least one compression zone of the first compression zone, the second compression zone, the third compression zone, the fourth compression zone, the fifth compression zone, the sixth compression zone, the seventh compression zone, the eighth compression zone, and the ninth compression zone includes
at least one ventilation zone seamlessly knitted into the jacquard knit stitch construction, wherein the at least one ventilation zone includes a mesh or pattern of drop stitch hole mesh textures.

11. The garment of claim 10, wherein the at least one ventilation zone includes a pattern of larger and smaller ventilation holes.

12. The garment of claim 10, wherein the ventilation zone is configured to improve breathability of the jacquard knit stitch construction, or is configured to provide a visual effect, or both.

13. A garment, comprising:
a pair of tights having a front side and a rear side, wherein the front side is sized and shaped to cover an area corresponding to a front portion of a wearer's torso and legs, wherein the rear side is sized and shaped to cover an area corresponding to a rear portion of a wearer's torso and legs, wherein the pair of tights has a left leg and a right leg, wherein the pair of tights comprises an engineered warp knitted component having a plurality of integrated compression zones, the plurality of integrated compression zones including:
a first compression zone on the front side, wherein the first compression zone is sized and shaped to cover at least a portion of a first area corresponding to a wearer's front torso below a waist, and wherein the first compression zone has a first amount of compression and a first fabric weight;
a second compression zone adjacent to the first compression zone, wherein the second compression zone is sized and shaped to cover at least a portion of a second area corresponding to a front side of a wearer's torso below a waist, and wherein the second compression zone has a second amount of compression and a second fabric weight;
a third compression zone adjacent to the second compression zone in each of the left leg and the right leg, wherein the third compression zone is sized and shaped to cover at least a portion of a third area corresponding to a wearer's leg at or below a torso and above a knee, wherein the third compression zone extends around the front side and around the rear side of each of the left leg and the right leg, and wherein the third compression zone has a third amount of compression and a third fabric weight; and
a fourth compression zone below the third compression zone in each of the left leg and the right leg, the fourth compression zone is sized and shaped to cover at least a portion of a fourth area corresponding to a wearer's knee, the fourth compression zone extends around the front side and around the rear side of each of the left leg and the right leg, and the fourth compression zone has a fourth amount of compression and a fourth fabric weight;
wherein the first compression zone is directly or indirectly joined to the second compression zone by a first seamless transition, the second compression zone is directly or indirectly joined to the third compression zone by a second seamless transition, and the third compression zone is directly or indirectly joined to the fourth compression zone by a third seamless transition;
wherein the first amount of compression is greater than the fourth amount of compression, or the second amount of compression is greater than the fourth amount of compression, or the third amount of compression is greater than the fourth amount of compression;
wherein a difference between the first fabric weight and the fourth fabric weight ranges from 100 grams per square meter (GSM) to 220 GSM, and wherein the first fabric weight is greater than the fourth fabric weight; and
wherein at least one compression zone of the first compression zone, the second compression zone, the third compression zone, and the fourth compression zone comprises a jacquard knit stitch construction, wherein the jacquard knit stitch construction includes an elastane jacquard construction, and the elastane jacquard construction is configured to increase a density or configured to increase a fabric weight or configured to increase both a density and a fabric weight.

14. The garment of claim 13, wherein the first amount of compression is about the same as the third amount of compression.

15. The garment of claim 13, further comprising a transition zone between the second compression zone and the third compression zone or between the third transition zone and the fourth compression zone, or between the second compression zone and the third compression zone and between the third compression zone and the fourth compression zone.

16. The garment of claim 15, wherein each transition zone includes at least one sub-compression zone, wherein the at least one sub-compression zone is a first sub-compression zone including a first A sub-compression zone immediately adjacent a first B sub-compression zone, the first A sub-compression zone has a first A amount of compression and a first A fabric weight, the first B sub-compression zone has a first B amount of compression and a first B fabric weight, the first A amount of compression and the first B amount of compression are different, the first A fabric weight and the first B fabric weight are different, and the first A fabric weight is greater than the first B fabric weight.

17. The garment of claim 16, further comprising a second sub-compression zone adjacent the first sub-compression zone, wherein the second sub-compression zone includes a second A sub-compression zone immediately adjacent a second B sub-compression zone, the second A sub-compression zone has a second A amount of compression and a second A fabric weight, the second B sub-compression zone has a second B amount of compression and a second B fabric weight, the first A amount of compression and the second A amount of compression are the same, and the first B amount of compression and the second B amount of compression are the same.

18. The garment of claim 13, further comprising a waistband affixed to the first compression zone of the engineered warp knitted component.

19. A garment, comprising:
a pair of tights having a front side and a rear side, wherein the front side is sized and shaped to cover an area corresponding to a front portion of a wearer's torso and legs, wherein the rear side is sized and shaped to cover an area corresponding to a rear portion of a wearer's torso and legs, wherein the pair of tights has a left leg and a right leg, wherein the pair of tights comprises an engineered warp knitted component having a plurality of integrated compression zones, the plurality of integrated compression zones including:
a first compression zone on the front side, wherein the first compression zone is sized and shaped to cover at least a portion of a first area corresponding to a wearer's front torso below a waist, and wherein the first compression zone has a first amount of compression and a first fabric weight;
a second compression zone adjacent to the first compression zone, wherein the second compression zone is sized and shaped to cover at least a portion of a second area corresponding to at least a back side of a knee joint of a wearer's leg but not a front side of a wearer's knee cap, wherein the second compression zone extends around the front side and around the rear side of each of the left leg and the right leg, and wherein the second compression zone has a second amount of compression and a second fabric weight; and
a third compression zone adjacent to the second compression zone in each of the left leg and the right leg, wherein the third compression zone is sized and shaped to cover a third area corresponding to a wearer's knee, wherein the third compression zone extends on the front side of each of the left leg and the right leg, and wherein the third compression zone has a third amount of compression and a third fabric weight;
wherein the first compression zone is directly or indirectly joined to the second compression zone by a first seamless transition, the second compression zone is directly or indirectly joined to the third compression zone by a second seamless transition, and the third compression zone is directly or indirectly joined to the fourth compression zone by a third seamless transition;
wherein the second amount of compression is greater than the first amount of compression and is greater than the third amount of compression;
wherein a difference between the second fabric weight and the first fabric weight or between the second fabric weight and the third ranges from 100 grams per square meter (GSM) to 220 GSM, and wherein the second fabric weight is greater than the first fabric weight or is greater than the third fabric weight or is greater than each of the first fabric weight and the third fabric weight; and
wherein at least one compression zone of the first compression zone, the second compression zone, and the third compression zone comprises a jacquard knit stitch construction, wherein the jacquard knit stitch construction includes an elastane jacquard construction, and the elastane jacquard construction is configured to increase a density or configured to increase a fabric weight or configured to increase both a density and a fabric weight.

20. The garment of claim 19, further comprising a waistband affixed to the first compression zone of the engineered warp knitted component.
